(19) United States
(12) Patent Application Publication
(10) Pub. No.: US 2006/0280701 A1
(43) Pub. Date: Dec. 14, 2006

(54) Method for dispersing metal oxides

(76) Inventor: Maurice Gerard Lynch, Waterloo (BE)

Correspondence Address: Paul A Fair;FMC Corporation, 1735 Market Street, Philadelphia, PA, 19103, US

(21) Appl. No.: 10/571,978

(22) PCT Filed: Sep. 20, 2004
(86) PCT No.: PCT/US04/30533

§ 371 Date: Mar. 15, 2006

Related U.S. Application Data
(60) Provisional application No. 60/504,029, filed on Sep. 18, 2003.

(51) Int. Cl. A61K 8/73; C08L 1/00; C08G 18/38; C09J 101/00

(52) U.S. Cl. 424/59; 424/70.13; 524/35; 977/926; 106/162.71

(57) ABSTRACT

The present invention relates to a method for preparing a stable dispersion of a metal oxide in water comprising dispersing colloidal microcrystalline cellulose in water either prior to or concurrently with adding the metal oxide and recovering the stable metal oxide dispersion, wherein: (i) the metal oxide has an average particle size of less than 250 nanometers, (ii) the metal oxide is not iron oxide, (iii) the colloidal microcrystalline cellulose is coprocessed with a polymeric binder and (iv) the metal oxide is present in an amount of at least 0.6% by weight of the total weight of the dispersion.

This application claims the benefit of U.S. Provisional Application No. 60/504,029, filed Sep. 18, 2003.

### FIELD OF THE INVENTION

The present invention relates to a method for dispersing metal oxides other than iron oxide. In another embodiment, the invention is a composition prepared by the process. In yet another embodiment, the invention is a cosmetic, sunscreen, pharmaceutical, paint, coating or food composition containing the composition prepared by the process. In yet another embodiment, the invention is a powder composition comprising colloidal microcrystalline cellulose and an inorganic UV filter metal oxide.

### BACKGROUND OF THE INVENTION

Metal oxides provide benefits in several applications. For example, iron oxides are widely used as pigments in the paint and coatings industry and in decorative cosmetics. Titanium dioxide is used as an opacifier or whitening agent in the paint and coatings industry and in cosmetics, food and pharmaceutical applications. Zinc oxide is used an active ingredient or opacifier in several cosmetic applications including decorative cosmetics and after-shave products.

Sunscreen compositions are applied to the skin to protect the skin from the sun's ultraviolet rays that produce erythema, a reddening of the skin commonly known as sunburn. Ultraviolet radiation (“UVR”) in the wavelength range of 290 nm to 320 nm (“UV-B”), which is absorbed near the surface of the skin, is the primary cause of sunburn. Ultraviolet radiation in the wavelength of 320 nm to 400 nm (“UV-A”) penetrates more deeply into the skin and can cause damaging effects that are more long term in nature. Prolonged and constant exposure to the sun may lead to actinic keratoses and carcinomas as well as to premature aging of the skin, characterized by skin that is wrinkled, cracked, and has lost its elasticity.

UV filters, which can be divided into two classes, organic and inorganic, are widely used to protect the skin or hair from the damaging effects of ultraviolet radiation. These UV filters can be formulated into various formats of cosmetic products including creams, lotions, sticks, gels and sprays.

Zinc oxide and titanium dioxide are particularly useful in sunscreen applications due to their ability to increase the sun protection factor (SPF) of formulations. In the past, the protective properties of these metal oxides were limited and their use resulted in a white residue on the skin. In recent years, substantial progress has been made in the development of more effective forms of both zinc oxide and titanium dioxide for sunscreens. This progress has involved the development of smaller particles of these metal oxides. Typically, when used in sunscreens, these inorganic UV filters have particle sizes of less than 100 nanometers. Inorganic UV filters are particularly valued for use in sunscreens and in other cosmetics because of the protection they provide over a broad range of UV wavelength. In addition, they are generally regarded as safe for cosmetic use, and do not have the disadvantage of a tacky skin-feel as is the case with most organic UV filters.

During the production of a sunscreen or cosmetic formulation, the inorganic UV filter can be dispersed in the oil phase or the water phase. Although the protective properties are improved by a reduction in particle size to below 100 nanometers, the dispersion of these metal oxides becomes more difficult. Failure to disperse the inorganic UV filter into individual particles will result in reduced absorbance of UV radiation because agglomerated particles have lower ability to absorb UV radiation than individual particles. Several methods are known that overcome this difficulty, either partially or completely. For example, the metal oxide can be dispersed in water using a dispersing aid such as polyhydroxy stearic acid. Although this method reduces the difficulty of dispersion, it introduces an additional ingredient that has no other function other than as a dispersing aid. Furthermore, dispersion aids known in the art do not provide stability against settling of the inorganic UV filter. Alternatively, the metal oxide can be dispersed in an oil, such as silicone oil. However, this process again introduces an additional ingredient and an additional step in the manufacturing process. This dispersion can then be added to the oil phase during the preparation of a sunscreen.

The manufacturers of sunscreens, or other cosmetics containing inorganic UV filters, can select from two different approaches with respect to how to incorporate inorganic UV filters in the formulation. The manufacturer can purchase a powder form of the inorganic UV filter and disperse this directly in the water phase or the oil phase. These powder forms of inorganic UV filters are readily available commercially and are often coated with substances to improve dispersibility. However, this coating and optionally the use of a dispersing aid only partially reduces the difficulty of dispersion. As a result, many manufacturers find that they encounter production issues with powder UV filters. A second approach involves the purchase of a predispersion of the inorganic UV filter in either an oil or in water. Although this approach largely overcomes the problem of dispersion, it is more costly for the manufacturer because the predispersion is typically more expensive than a powder product. In addition, the manufacturer has to choose from a limited number of available predispersion compositions.

Therefore, there is a need for a convenient method of dispersing organic UV filters directly into sunscreen and other formulations that avoids the need for predispersions. In addition, there is a need to improve stability of aqueous dispersions of organic UV filters.

### SUMMARY OF THE INVENTION

The present invention relates to a method for preparing a stable dispersion of a metal oxide in water comprising dispersing colloidal microcrystalline cellulose in water either prior to or concurrently with adding the metal oxide and recovering the stable metal oxide dispersion, wherein: (i) the metal oxide has an average particle size of less than 250 nanometers, (ii) the metal oxide is not iron oxide, (iii) the colloidal microcrystalline cellulose is coprocessed with a polymeric binder and (iv) the metal oxide is present in an amount of at least 0.6% by weight of the total weight of the dispersion This stable dispersion can then be packaged, sold and used at a later date to finish the formulation. In another embodiment, the invention is a composition prepared by the process. In yet another embodiment, the invention is a cosmetic, pharmaceutical, paint, coating or food composition containing the composition prepared by the process.

We have discovered that the metal oxides of the invention can be readily dispersed in water when dispersion of the metal oxide is either after or concurrent with the dispersion of colloidal microcrystalline cellulose in said water. The dispersed metal oxide remains stable during storage and can be readily incorporated into a sunscreen or other cosmetic formulation at a later date.

### DETAILED DESCRIPTION OF THE DRAWINGS

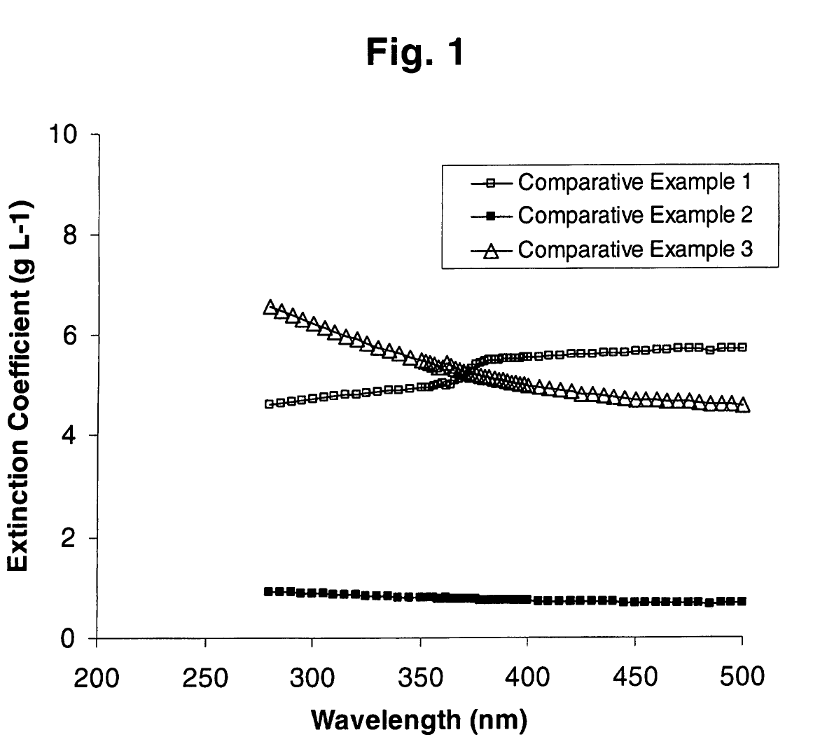

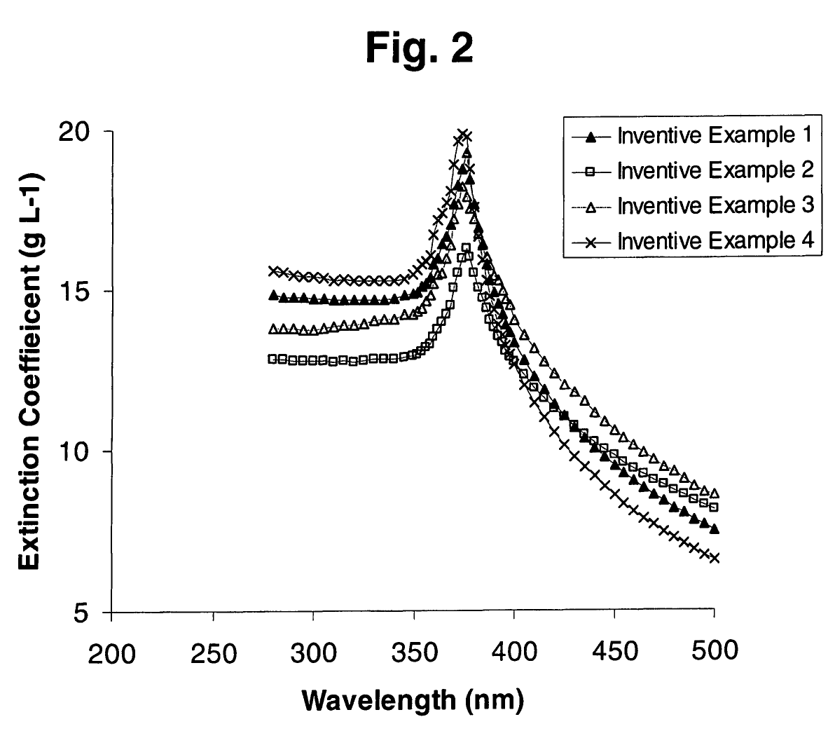

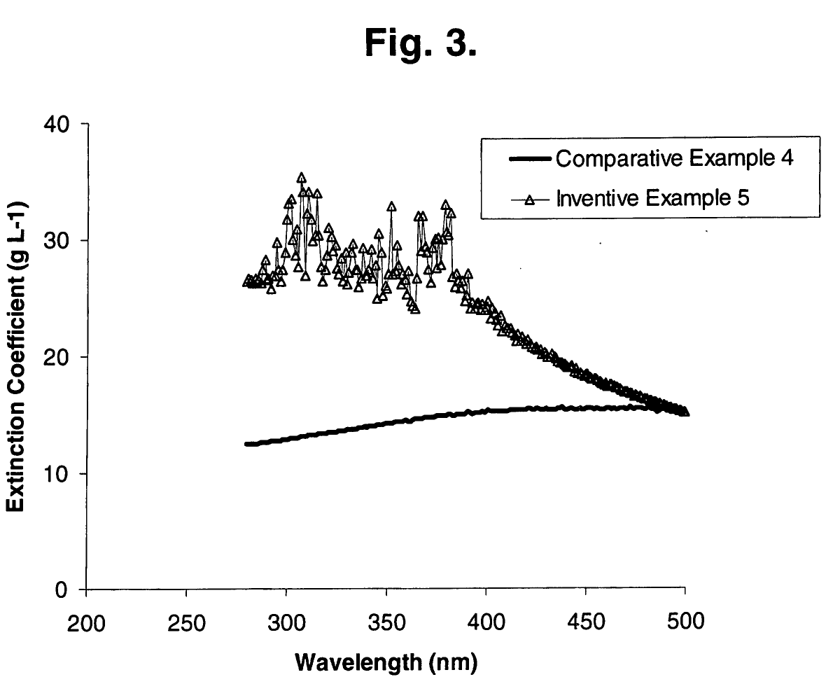

FIGS. 1 and 2 show plots of extinction coefficient versus wavelength for Comparative Examples 1 to 3 and Inventive Examples 1 to 4. FIG. 3 shows plots of extinction coefficient versus wavelength for Comparative Example 4 and Inventive Example 5.

### DETAILED DESCRIPTION OF THE INVENTION

Microcrystalline cellulose is a purified, partially depolymerized cellulose that is produced by treating a source of cellulose, preferably alpha cellulose, in the form of a pulp from fibrous plants, with a mineral acid, preferably hydrochloric acid. The acid selectively attacks the less ordered regions of the cellulose polymer chain, thereby exposing and freeing the crystallite sites, forming the crystallite aggregates that constitute microcrystalline cellulose.

Colloidal microcrystalline cellulose is obtained by reducing the particle size of microcrystalline cellulose by attrition and stabilizing the attrited particles to avoid formation of hard aggregates. The method of drying may be any method which ultimately produces a reconstitutable powder. One such method is spray drying, which can be used to produce microcrystalline cellulose coprocessed with a polymeric binder such as sodium carboxymethylcellulose, carrageenan, alginate, pectin and pectates, and xanthan. Techniques for reducing the particle size of microcrystalline cellulose and/or for spray drying microcrystalline cellulose are disclosed in Durand, U.S. Pat. No. 3,539,365; Krawczyk, U.S. Pat. No. 6,025,037; Venables, U.S. Pat. No. 6,037,080, and Tuason, U.S. Pat. No. 6,392,368. As long as sufficient colloidal microcrystalline cellulose is present in the composition to control rheology, the composition may also comprise larger microcrystalline particles, for example, particles that have not been attrited or only partially attrited, provided the composition does not become grainy.

The colloidal microcrystalline cellulose of the invention is coprocessed with a polymeric binder. Such polymeric binders include the sodium salt of carboxymethylcellulose.

Colloidal microcrystalline celluloses comprising microcrystalline cellulose and sodium salt of carboxymethylcellulose are commercially available. AVICEL® RC-581 and AVICEL® RC-591 each contain microcrystalline cellulose and sodium carboxymethylcellulose in a ratio of approximately 89/11, by weight. AVICEL® CL-611 and AVICEL® PC-611 each contain microcrystalline cellulose and sodium carboxymethylcellulose in ratio of approximately 85/15, by weight. Preferred colloidal microcrystalline celluloses are AVICEL® CL-611 and AVICEL® PC-611. Colloidal particle size is generally less than about 1 micron.

Colloidal microcrystalline cellulose forms a three dimensional structuring network when dispersed in water. Dispersion is achieved by adding microcrystalline cellulose, which is typically available commercially as a powder, to water and applying sufficient shear to cause separation of individual microcrystals. It is critical to the current invention that the colloidal microcrystalline cellulose be at least partially dispersed in the water. To verify that the colloidal microcrystalline cellulose is partially dispersed, a sample of the dispersion can be viewed under a microscope using polarized light and a magnification of 100×. If the microcrystals are properly dispersed they will appear as individual white specks homogeneously distributed on a black background.

The colloidal microcrystalline cellulose may be present in an amount of from about 0.1% to about 5%, more particularly, about 0.2% to about 2%, by weight of the total weight of the dispersion.

The metal oxide of the current invention is any metal oxide excluding iron oxide that finds utility in sunscreens, cosmetics, personal care, pharmaceutical, paint, coatings, food and printing applications. Examples of metal oxides include titanium dioxide and zinc oxide. Preferred oxides include titanium dioxide and zinc oxide. Preferred titanium dioxides include those with average particle sizes of less than less than 250 nanometers, less than 200 nanometers, preferably less than 100 nanometers, when fully dispersed. Examples of suitable titanium dioxides include those sold under the tradename UV-Titan by Kemira. The titanium dioxide may be treated with a surface coating to prevent a photo oxidation reaction on the skin. The titanium dioxide may further be treated with a surface material to improve dispersibility in either water or oil, such as hydrophobic, hydrophilic or neutral coatings. Preferred zinc oxides include those with average particle sizes of less than 250 nanometers, less than 200 nanometers, preferably less than 100 nanometers, when fully dispersed. Examples of suitable zinc oxides include those sold commercially under the tradename Z-Cote by BASF and under the tradename Zinc Oxide Neutral by Haarmann and Reimer. The zinc oxide may be treated with a surface material to improve dispersibility in either water or oil, e.g., hydrophobic, hydrophilic and neutral coatings. Preferably the metal oxide is a powder.

As used herein, all amounts indicated by % are by weight of the total dispersion including water unless specifically indicated otherwise.

The amount of metal oxide used in the dispersion of the present invention including water is at least 0.6% by weight of the total weight, more particularly, from about 0.6% to about 99% by weight of the total weight, more particularly from about 0.6% to about 80% by weight of the total weight, more particularly, from about 0.6% to about 50% by weight of the total weight, more particularity, from about 1% to about 50% by weight of the total weight, more particularly, from about 2% to about 40% by weight of the total weight. The metal oxide may be present in an amount of about 10% by weight of the total weight.

In one embodiment, the invention is a method for dispersing metal oxides excluding iron oxides comprising dispersing the metal oxide excluding iron oxide in water either after or concurrent with dispersion of the colloidal microcrystalline cellulose.

In another embodiment, the invention is the product of the method of the invention. This product can then be packaged and sold for finishing the desired formulation at a later date.

In a further embodiment, the invention is a sunscreen, a cosmetic, pharmaceutical, food, textile, paint or coating composition formulated from the product of the process of the invention. Both the stable dispersion of the invention and formulations made from the stable dispersion can contain any of an organic sunscreen, a dispersing aid, an emollient, a surfactant, a color, an humectant, a secondary stabilizer, a preservative, an active ingredient, a film former, a fixative, or a water-proofing agent. Examples of the secondary stabilizer include a synthetic polymer such as an acrylate, polyvinylpyrrolidone and modified carboxymethylcellulose; and polysaccharides such as alginate, carrageenan, pectin, guar and xanthan gum. The secondary stabilizer is generally present in an amount of from 0-3 wt % of the total weight, more particularly, 0.075-0.5 wt % of the total weight.

In yet another embodiment, the invention is a powder composition made by the process of the invention. The amount of the colloidal microcrystalline cellulose present in the powder composition of the invention is from about 1% to about 200% by weight of the metal oxide, preferably from about 5% to about 100% by weight of the metal oxide, more preferably from about 10% to about 50% by weight of the metal oxide.

The process of the invention, and the products thereof, desirably have applications in the manufacture of sunscreens. It provides a cost-effective method for the incorporation of inorganic UV filter by allowing a sunscreen manufacturer to utilize cheaper powder forms of inorganic UV filters and avoid the need for more expensive predispersions. Similar applications exist in the manufacture of other cosmetic products. UV filters are increasingly used in a wide variety of cosmetics to provide protection from the harmful effects of sunlight. Examples of such cosmetics include day creams, sunless tanning preparations, hair care products and decorative cosmetics, including lipsticks, mascaras, face powders, eye shadows, eye liners and lip glosses. Further applications exist in the paint and coating industry, especially in automotive coatings, and in the pharmaceutical, food and textile printing industries.

The powder composition of the invention will find utility in the preparation of sunscreens and other cosmetic products. The advantageous properties of this invention can be observed by reference to the following examples, which illustrate but do not limit the present invention.

### EXAMPLES

### Materials

In all cases the water used was deionized water.

| INCI name | Tradename | Supplier | Function |
|---|---|---|---|
| Microcrystalline | Avicel ® | FMC | Stabilizing |
| Cellulose (and) | PC 611 | BioPolymer | agent |
| Cellulose Gum^{a} | | | |
| Carrageenan | Gelcarin | FMC | secondary |
| | GP 911 | BioPolymer | stabilizer |
| Xanthan | | | secondary |
| | | | stabilizer |
| Sodium Benzoate | | | preservative |
| Diazolidinyl urea | Germall Plus | ISP | preservative |
| (and) Iodopropynyl | | | |
| butylcarbamate | | | |
| Titanium Dioxide | UV-Titan M212 | Kemira | Sunscreen |
| (and) Glycerin | | | |
| (and) Alumina | | | |
| Titanium Dioxide | UV-Titan M170 | Kemira | Sunscreen |
| (and) Alumina | | | |
| (and) Methicone | | | |
| Zinc Oxide | Zinc Oxide | Haarmann | Sunscreen |
| | Neutral | & Reimer | |

In all Examples, except where otherwise stated, the term ‘dispersing’ refers to the following procedure. During a time period of approximately 30 seconds, powder ingredients were added slowly to water while mixing with a Silverson rotor-stator mixer at low speed (2,000 rpm). After all the powder had been added, the dispersion was mixed for 10 minutes at high speed (8,000 rpm) then stored at room temperature (approximately 20° C.) until evaluation. All concentrations are % (w/w).

### Comparative Example 1

This Comparative Example illustrates that the UV absorbing ability of zinc oxide is low if it is dispersed into water alone (i.e., not containing or dispersed with the colloidal microcrystalline cellulose) by the procedure above. A dispersion containing 3.5% zinc oxide was prepared by dispersing 17.5 g of Zinc Oxide Neutral in 482.5 g of water.

### Comparative Example 2

This Comparative Example illustrates that microcrystalline cellulose has very low ability to absorb UV radiation. A dispersion of 1.5% microcrystalline cellulose was prepared by dispersing 7.5 g of Avicel® CL 611 to 492.5 g of water.

### Comparative Example 3

This Comparative Example illustrates that microcrystalline cellulose has no impact on the UV absorbing ability of zinc oxide if microcrystalline cellulose and zinc oxide are dispersed in separate portions of water that are later combined. A 7% dispersion of zinc oxide was prepared by dispersing 35 g of Zinc Oxide Neutral in 465 g of water. A dispersion of 3% microcrystalline cellulose was prepared by dispersing 15 g of Avicel® CL 611 in 485 g of water. A mixed dispersion containing 3.5% zinc oxide and 1.5% microcrystalline cellulose was prepared by adding 250 g of the 7% zinc oxide dispersion to 250 g of the 3% microcrystalline cellulose dispersion and mixing for 3 minutes with a propeller mixer at low speed (300 rpm).

### Inventive Example 1

This Example illustrates that improved dispersion of zinc oxide occurred when microcrystalline cellulose powder was blended with zinc oxide powder prior to dispersing in water. A powder blend of zinc oxide/microcrystalline cellulose was prepared by mixing 17.5 g of Zinc Oxide Neutral and 7.5 g of Avicel® CL 611 and shaking vigorously in a closed plastic pot for 3 minutes. A dispersion containing 3.5% zinc oxide and 1.5% microcrystalline cellulose was then prepared by dispersing the powder blend in 475 g of water.

### Inventive Example 2

This Example illustrates that improved dispersion of zinc oxide occurred when zinc oxide was dispersed into a dispersion of microcrystalline cellulose. A dispersion of 3.5% zinc oxide and 1.5% microcrystalline cellulose blend was prepared by first dispersing 7.5 g Avicel® CL 611 in 475 g of water and then dispersing 17.5 g of zinc oxide.

### Inventive Example 3

This Example also illustrates that improved dispersion of zinc oxide occurred when zinc oxide was dispersed into a dispersion of microcrystalline cellulose. A dispersion of 3.5% zinc oxide and 0.5% microcrystalline cellulose blend was prepared by first dispersing 2.5 g Avicel® CL 611 in 480 g of water and then dispersing 17.5 g of zinc oxide.

### Inventive Example 4

This Example illustrates that improved dispersion of zinc oxide occurred when zinc oxide was coprocessed with microcrystalline cellulose before dispersion in water. A coprocessed mixture of zinc oxide and microcrystalline cellulose was prepared by mixing Zinc Oxide Neutral and Avicel® CL 611 in water, at a w/w ratio of 30 parts Avicel® CL 611 to 70 parts Zinc Oxide Neutral, using high shear and then drying using a spray drier.

A dispersion of 3.5% zinc oxide and 1.5% microcrystalline cellulose was prepared by dispersing 25 g of the coprocessed mixture in 475 g of water.

### Comparative Example 4

This Comparative Example illustrates that the UV absorbing ability of titanium dioxide is low if it is dispersed into water. A dispersion containing 3.5% titanium dioxide was prepared by dispersing 17.5 g of UV-Titan M212 in 482.5 g of water.

### Inventive Example 5

This Example illustrates that improved dispersion of titanium dioxide occurred when titanium dioxide was dispersed into a dispersion of microcrystalline cellulose. A dispersion of 3.5% titanium dioxide and 1.5% microcrystalline cellulose blend was prepared by first dispersing 7.5 g Avicel® CL 611 in 475 g of water and then dispersing 17.5 g of UV-Titan M212.

### Methods of Evaluation

### Stability Testing

Samples of the dispersions prepared in the Examples were stored at room temperature (approximately 20° C.). Samples were determined to be stable if no visible sediment was present after 1 month.

### Stability results

Comparative Examples 1 and 4 was not stable. Comparative Examples 2 and 3 and Inventive Examples 1-5 were all stable. These results indicate the ability of colloidal microcrystalline cellulose to stabilize dispersions of zinc oxide and dispersions of titanium dioxide.

### Spectrophotometric Testing

Spectrophotometry was used to determine if the UV filters were well dispersed. Improved dispersion would result in higher absorbance in the UV region. The dispersions prepared in the Examples were diluted by adding 2 g of dispersion to 248 g of water and mixing gently with a magnetic stirrer for 3 minutes. The absorbance of these diluted dispersions was measure at 1 nm intervals at wavelengths between 280 and 500 nm using a quartz cuvets, pathlength 1 cm, and a Hewlett Packard 8453 spectrophotometer. The Extinction Coefficient at each wavelength was calculated as absorbance divided by concentration (g L-1).

### Spectrophotometric Results

FIGS. 1 and 2 show plots of extinction coefficient versus wavelength for Comparative Examples 1 to 3 and Inventive Examples 1 to 4. Increased absorbance in the UV region indicates that zinc oxide is better dispersed in Inventive Examples 1 to 4 compared to Comparative Examples 1 and 3. Comparative Example 2 shows that colloidal microcrystalline cellulose absorbs very little radiation in the UV region.

FIG. 3 shows plots of extinction coefficient versus wavelength for Comparative Example 4 and Inventive Example 5. Increased absorbance in the UV region indicates that titanium dioxide is better dispersed in Inventive Example 5 compared to Comparative Example 4.

### Inventive Example 6

The dispersion compositions of Tables 1 and 2 were prepared as follows. AVICEL® PC-611 was dispersed in water using a Silverson homogeniser at 3500 rpm followed by 10 minutes mixing. If a secondary stabilizer was used, it was added and stirred for 5 minutes. The preservative was added and stirred for 3 minutes. The pigment was added portion wise to the vortex over approximately an hour while mixing. The mixing speed was increased to 5000 to 8000 rpm as required to maintain a vortex. The suspension was mixed for 10 minutes after the final addition. Mixing was then stopped and the sample was de-aerated by applying a vacuum of 0.1 mm Hg to the dispersion sample for 10 minutes to remove entrained gas. The sample was then placed in a storage container and stored overnight at room temperature. Brookfield viscosity of the sample was measured the next day. Samples were stored at room temperature (˜20° C.), 40° C. and 50° C. Samples were tested weekly. Samples were allowed to return to room temperature before examination. All samples remained stable to separation for eight weeks of testing, i.e., no liquid separation or visible particle separation.

TABLE 1

| Ingredients | 6-1 | 6-2 | 6-3 | 6-4 | 6-5 |
|---|---|---|---|---|---|
| AVICEL ® PC-611 | 3.0 | 3.0 | 3.0 | 3.0 | 4.0 |
| UV Titan M170 | 15 | 20 | 20 | 25 | 30 |
| Xanthan | 0 | 0 | 0.15 | 0.15 | 0.15 |
| Sodium benzoate | 0.3 | 0.3 | 0 | 0.3 | 0.3 |
| Germall plus | 0 | 0 | 0.2 | 0 | 0 |
| Deionized water | 81.7 | 76.7 | 76.65 | 71.55 | 65.55 |
| Viscosity, mPs | 48,000 | 85,200 | 210,500 | 322,000 | 516,000 |
| Spindle, | #2, 26 | #3 25 | 6, 25 | #6 26 | #6, 26 |
| Temp(° C.) | | | | | |
| RT stability | Ok | Ok | Ok | Ok | Ok |
| 40 C. stability | Ok | Ok | Ok | Ok | Ok (Y) |
| 50 C. stability | Ok | Ok | Ok | Ok | Ok |

TABLE 2

| Ingredient | 6-6 | 6-7 | 6-8 | 6-9 |
|---|---|---|---|---|
| AVICEL ® PC-611 | 2 | 2 | 2 | 2 |
| UV Titan M170 | 15 | 15 | 20 | 20 |
| Gelcarin GP911 | 0 | 0.2 | 0 | 0.2 |
| Sodium benzoate | 0.3 | 0.3 | 0.3 | 0.3 |
| Deionized Water | 82.5 | 82.5 | 77.5 | 77.5 |
| Viscosity, mPas | 40,240 | 72,400 | 120,700 | 170,000 |
| Spindle, | #2/26 | #3/26 | #3/26 | #4/26 |
| Temp(° C.) | | | | |
| RT Stability | Ok (Y) | Ok (Y) | Ok (Y) | Ok |
| 40° C. stability | Ok | Ok | Ok | Ok |
| 50° C. stability | OK | Ok | Ok | Ok |

What is claimed is:

1. A method for preparing a stable dispersion of a metal oxide in water comprising dispersing colloidal microcrystalline cellulose in water either prior to or concurrently with adding said metal oxide and recovering said stable metal oxide dispersion, wherein: (i) said metal oxide has an average particle size of less than 250 nanometers, (ii) said metal oxide is not iron oxide, (iii) said colloidal microcrystalline cellulose is coprocessed with a polymeric binder and (iv) said metal oxide is present in an amount of at least 0.6% by weight of the total weight of the dispersion.

2. A method according to claim 1 wherein the metal oxide comprises at least one member selected from the group consisting of titanium dioxide and zinc oxide.

3. A method according to claim 2 wherein the metal oxide comprises an inorganic UV filter selected from titanium dioxide and zinc oxide.

4. A method according to claim 1 wherein the colloidal microcrystalline cellulose is present in an amount of from about 0.1% to about 5% by weight and the metal oxide is present from about 0.6% to about 50% by weight.

5. A method according to claim 1 wherein the colloidal microcrystalline cellulose is present in an amount of from about 0.2% to about 2% by weight and the metal oxide is present from about 2% to about 40% by weight.

6. A dispersion composition comprising colloidal microcrystalline cellulose and a metal oxide prepared according to claim 1.

7. A dispersion composition according to claim 6 further comprising a preservative.

8. A composition comprising the dispersion composition of claim 6.

9. A composition according to claim 8 wherein said composition is a cosmetic, sunscreen, pharmaceutical, paint, coating, textile or food product.

10. A powder composition comprising colloidal microcrystalline cellulose and an inorganic UV filter metal oxide comprising at least one of titanium dioxide and zinc oxide, wherein: (i) said metal oxide has an average particle size of less than 250 nanometers, (ii) said metal oxide is not iron oxide, (iii) said colloidal microcrystalline cellulose is coprocessed with a polymeric binder and (iv) said metal oxide is present in an amount of at least 0.6% by weight of the total weight of the dispersion.

11. A powder composition according to claim 10 wherein the amount of the colloidal microcrystalline cellulose is present in an amount of from about 1% to about 200% by weight of the inorganic UV filter.

12. A powder composition according to claim 11 wherein the amount of the colloidal microcrystalline cellulose is from about 5% to about 100% by weight of the inorganic UV filter.

13. A powder composition according to claim 12 wherein the amount of the colloidal microcrystalline cellulose is from about 10% to about 50% by weight of the inorganic UV filter.

14. The method of claim 1 wherein a secondary stabilizer is added to the dispersion prior to adding said metal oxide.

15. The method of claim 14, wherein said secondary stabilizer comprises at least one member selected from the group consisting of synthetic polymers and polysaccharides.

16. The method of claim 15, wherein said synthetic polymer comprises at least one member selected from the group consisting of an acrylate, polyvinylpyrrolidone and modified carboxymethylcellulose, and said polysaccharide comprises at least one member selected from the group consisting of carrageenan, alginate, pectin, guar, pullulan and xanthan gum.

17. The method of claim 1, wherein said metal oxide is coated with a hydrophobic surface coating.

18. The method of claim 1, wherein said metal oxide has an average particle size of 200 nanometers or less.

19. The method of claim 1 wherein said colloidal microcrystalline cellulose and said metal oxide are coprocessed together prior to dispersion.

20. The method of claim 1, wherein said metal oxide has an average particle size of 100 nanometers or less.
